(19) United States
(12) Patent Application Publication
(10) Pub. No.: US 2026/0229372 A1
(43) Pub. Date: Aug. 6, 2026

(54) VALID MESSAGE INTERVAL FOR PATIENT MESSAGING INBOX

(71) Applicant: Medtronic, Inc., Minneapolis, MN (US)

(72) Inventors: Charles R. Stomberg, Forest Lake, MN (US); Gaurav Makin, North Oaks, MN (US); Benjamin Thomas Jeannot, Blaine, MN (US)

(21) Appl. No.: 19/149,670

(22) PCT Filed: Jan. 18, 2024
(86) PCT No.: PCT/IB2024/050486

§ 371 Date: Jul. 21, 2025

Related U.S. Application Data
(60) Provisional application No. 63/481,448, filed on Jan. 25, 2023.

(51) Int. Cl. G16H 80/00; H04L 51/224

(52) U.S. Cl. CPC G16H 80/00; H04L 51/224

(57) ABSTRACT

An example system includes at least one device comprising a memory configured to store medical device data generated by a medical device of a patient, and one or more programmable processors in communication with the memory. The one or more programmable processors are configured to determine, based on the medical device data, a message for a user and an expiration time stamp for the message after which the message is no longer valid, and provide the message including the expiration time stamp to a user computing device.

This application claims the benefit of U.S. Provisional Patent Application Ser. No. 63/481,448, filed Jan. 25, 2023, the entire content of which is incorporated herein by reference.

### TECHNICAL FIELD

The present application relates to systems including medical devices and, more particularly, to providing medical messages using such systems.

### BACKGROUND

In many situations, a medical device, such as an implantable medical device (IMD), partially implantable medical device or wearable medical device, or other patient monitoring device may include wireless connectivity features that enable communication with computing devices such as programmers, patient monitors, and smartphones of the patient, and/or cloud-based computing systems. Medical devices may transmit a variety of information to such computing devices and systems, including physiological data of the patient and operational data of the medical device. Additionally, the medical device or other computing devices or systems may have the capability to provide messages to patients and/or medical professionals. The messages may be notifications regarding operation of the medical device, detection or prediction of a medical event based on physiological data collected by the medical device, or surveys intended to collect medical information from the patient. In some examples, a medical device may include a companion application for the patient, e.g., on the patient's smartphone, to check messages related to their medical device and/or health.

### SUMMARY

Generally, this disclosure describes techniques performed by a computer system for processing medical device data and generating timed patient message. A medical device, such as an IMD, may automatically generate medical device data and provide it to a remote medical system via a patient's device, such as a smartphone. The medical device data may include therapy use statistics, diagnostic data, impedance, heart rate, heart rate variance, digitized sensed signals (e.g., for sensed arrhythmias or other events), device status, device history, and other data. The remote medical system may compare the received medical device data or a classification of the data determined by the system with a database containing a plurality of messages and associated categories to determine a message for a user, e.g., the patient, based on the medical device data. The remote medical system may determine an expiration time stamp representing a time duration for the message based on the category of message. The remote medical system may provide the message and the expiration time stamp to a user computing device. The user's computing device may make the message available, e.g., display or present the message, to the user until the time elapsed exceeds the time duration associated with the time stamp.

Patients may receive numerous messages, e.g., from a cloud monitoring system, from their clinic, and from medical devices implanted in their body. Sometimes, patients may not notice a message until it is no longer relevant but are still required to view and dismiss the irrelevant message. In other cases, patients may not quickly notice a message providing notification of a potentially life-threatening event if that message is mixed in with a number of other, less serious messages. Associating messages with a predetermined length of validity, e.g., using time stamps as described herein, helps patients manage messages relating to their medical device by reducing the total number of messages and ensuring that only relevant messages are shown. For example, a patient may not regularly check their computing device for messages regarding their medical device. When the patient does check their messages, they may be overwhelmed by the number and may struggle to determine which messages remain relevant. The addition of time durations to the message may ensure such a patient only sees messages that remain relevant. Additionally, associating message with an “urgent” banner or other indicator can help patients more quickly notice high priority messages.

In one example, this disclosure describes a system including at least one device comprising memory configured to store medical data generated by a medical device of a patient and one or more programmable processors in communication with the memory and configured to determined, based on the medical device data, a message for a user and an expiration time stamp for the message after which the message is no longer valid and provide the message including the expiration time stamp to a user computer device.

In another example, this disclosure describes a method comprising determining, based on medical device data generated by a medical device of a patient, a message for a user and an expiration time stamp for the message after which the message is no longer valid and providing the message including the expiration time stamp to a user computer device of the user.

In another example, this disclosure describes a non-transitory computer-readable storage device comprising instructions for a causing processing circuitry to determine, based on medical device data generated by a medical device of a patient, a message for a user and an expiration time stamp for the message after which the message is no longer valid and provide the message including the expiration time stamp to a user computing device of the user.

In another example, this disclosure describes a computing device comprising communication circuitry configured to receive a message and an expiration time stamp of the message from another device, wherein the expiration time stamp indicates a time after which the message is no longer valid, the message and the expiration time stamp determined by the other device based on medical device data of a medical device of a patient. The computing device further comprises a user interface and one or more programmable processors configured to control presentation of the message via the user interface based on the expiration time stamp.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example medical device system configured for communication of medical device data from an implantable medical device and messages based on the medical device data according to the techniques of the present disclosure.

FIG. 2 is a block diagram illustrating an example configuration of a medical device that operates in accordance with the one or more techniques of the present disclosure including generating and communicating medical device data.

FIG. 3 is a block diagram illustrating an example configuration of a computing device that operates in accordance with the one or more techniques of the present disclosure.

FIG. 4 is a block diagram illustrating an example configuration of a health monitoring system that operates in accordance with one or more techniques of the present disclose.

FIG. 5 is a flow diagram illustrating an example technique for determining a message and time stamp for the message based on medical device data collected by a medical device of a patient.

### DETAILED DESCRIPTION

A variety of types of medical devices, such as implantable medical devices (IMDs), are configured to sense physiological signals and generate data associated with patient metrics and/or provide therapy to a patient. These medical devices may provide the patient metric data and other data, e.g., relating to delivery of therapy or other performance metrics of the medical device, to external devices and systems. External devices may include smartphones, desktop, laptop, dedicated patient monitors, tablet computers, or workstations associated with such systems or entities, or employees of such systems or entities, or the patient. Such external devices may facilitate monitoring the patient's health, the functioning of their medical device, or other information associated with the patient or their respective medical device(s), and provide such data to remote or cloud-based health systems, such as cloud platforms for medical diagnosis/monitoring, which may be associated with the medical device(s) of the patient.

Remote health systems may provide messages about patient health or operation of a patient's medical device to a user computing device of the patient or a clinician or caregiver for the patient, such as a smartphone. For example, a patient may utilize an application on their smartphone or other computing device as a companion application for their IMD, and may receive messages relating to their medical device or otherwise related their medical care via the application. Clinicians or other users may similarly receive messages regarding the patient from a remote health system based on medical device data from the patient's medical device. In some examples, a patient may receive messages directly from their medical device using their computing device. In some examples, the remote system or the medical device may determine that a medical event is occurring or predicted to occur, and responsively generate a message. A patient may receive a variety of messages regarding their health such as reminders to visit a clinic, warnings about medical data generated by a patient's IMD, and other messages through the application.

Medical devices may sense and monitor one or more physiological parameters, detect status of health conditions of the patient that may indicate conditions, such as tachyarrhythmias and other arrhythmias, heart failure (HF) and recovery from HF, glucose levels, blood alcohol levels, respiration rates, tachycardia prediction, blood oxygen levels, other medical statistics, and/or provide therapy to the patient. The medical devices may be an implantable medical device (IMD), a partially implantable medical device, or a wearable medical device. Example IMDs in the cardiac space include pacemakers, implantable cardioverter-defibrillators (ICDs), cardiac resynchronization therapy (CRT) devices, which may be coupled to intravascular or extravascular leads, as well as pacemakers or pulmonary artery pressure sensors having housings configured for implantation intracardiac or intravascular, and implantable or wearable cardiac monitors, wearable cardiac defibrillators, or other wearable devices (e.g., smartwatches) that are capable of monitoring cardiac activity of a patient. The techniques of this disclosure may also be used in non-cardiac applications, such as with medical devices such as drug pumps, neurostimulators (e.g., brain stimulators, spinal cord stimulators, pelvic stimulators or the like), and many other types of wearable, implantable or partially implantable medical devices. Such medical devices may facilitate relatively longer-term monitoring of patients during normal daily activities and may periodically transmit collected data to a remote patient monitoring system, such as the Medtronic Carelink™ Network.

A medical device, e.g., a pacemaker, cardioverter and/or defibrillator, or a monitor that does not provide intervention, may generate and store patient data regarding patient metrics. The patient metrics may include, as examples, fluid level, heart rate, respiration rate, patient activity, temperature, heart sounds, oxygenation, and R-wave morphological characteristics. Other example patient metrics include coughing, speech, posture, tissue perfusion, hematocrit, thoracic impedance, subcutaneous impedance, intracardiac impedance, heart rate variability, weight, blood pressure, sleep apnea burden (which may be derived from respiration rate), ischemia burden, sleep duration, sleep quality, PVC burden, the occurrence, frequency, or duration cardiac events, and sensed cardiac intervals (e.g., heart rate or Q-T intervals). Examples of cardiac events may include atrial and ventricular tachyarrhythmias, atrial fibrillation, ventricular rate during atrial fibrillation, fluid level, heart rate, respiration rate, patient activity, temperature, heart sounds, oxygenation, and R-wave morphological characteristics. The concentration or levels of various substances, such as blood glucose, hematocrit, troponin and/or brain natriuretic peptide (BNP) levels, within the patient may also be used as one or more patient metrics.

This disclosure describes techniques for generating messages to a user, e.g., a patient, based on medical device data from the patient's IMD, with at least one time stamp that is indicative of the length of time the message is valid. The length of time may be determined by a category for the message, e.g., urgent-short, urgent-long, medium, low, etc., selected based on the medical device data, e.g., a category of a medical event indicated by data generated by the IMD. In some implementations, the category of medical of event would correspond to a category of message. More particularly, this disclosure describes techniques for determining, based on medical data from an IMD and a categorization database, a message category. In some examples, the message category may be a medical event category such as a stroke, heart attack, seizure, blood sugar level, and other such medical categories. In some examples, the medical event category can include an indicator of particularly high severity and/or risk such a life-threatening condition.

The message category may be used to determine the length of time that a message to a patient may be useful. In one example, a message to a patient warning of a seizure that is about to occur is of minimal use several weeks after the seizure occurs. In another example, a message about an ongoing medical condition may be valid for several weeks after the condition is first detected by the IMD. Using the message category, the remote medical system, IMD, or other message generating device may determine the period for which a message to a patient's device should remain valid. Further, the message generating device may configure the message to the patient's device such that the message contains a time stamp after which the message is no longer valid.

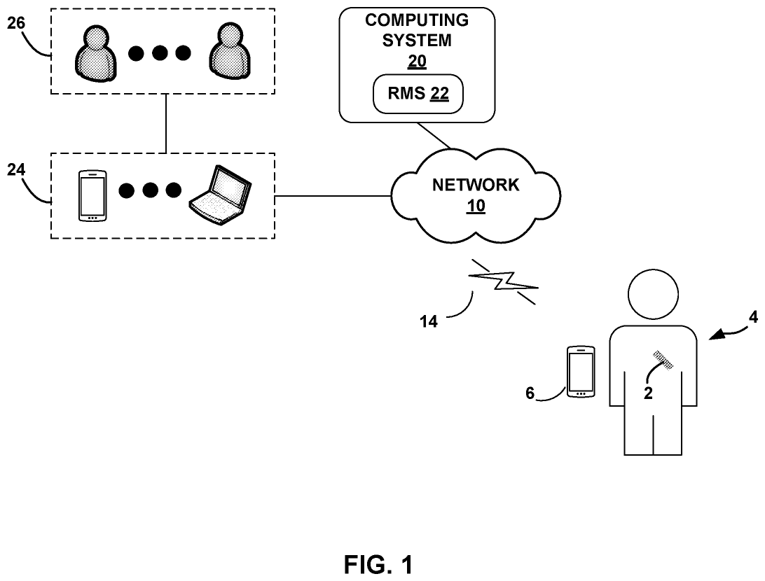

FIG. 1 is a block diagram illustrating an example system configured to report patient data from IMD 2 to network 10 and provide messages to user computing device 6. The example techniques may be used with one or more patient medical devices, e.g., IMD 2, which may be in wireless communication with one or more patient computing devices, e.g., user computing device 6. User computing device 6 may be any one of a computing device such as a smartphone, PDA, tablet computer, laptop computer, or any other such computing device. User computing device 6 may be utilized by patient 4, patient 4's caregiver, family member, or another person associated with patient 4. IMD 2 may communicate wirelessly with user computing device 6 and provide data such as patient metrics to user computing device 6. User computing device 6 may communicate with network 10 via communications 14. Communications 14 may include one or more communications mediums or protocols such as Wi-Fi®, Ethernet, cellular communications, fiber optic, and other such manners of communication. Although not illustrated in FIG. 1, IMD 2 include electrodes and other sensors to sense physiological signals of patient 4 to detect patient metrics and may collect and store detected patient metrics.

IMD 2 may generate data consistent with a patient experiencing a medical event. The data from IMD 2 may be provided via user computing device 6 to computing system 20 connected to network 10. Computing system 20 may include cloud computing, virtualized computing, distributed computing, servers, and other such computing equipment. Additionally, a remote medical system, RMS 22, may execute on computing system 20. RMS 22 may include a medical categorization database that stores a plurality of medical conditions and associated symptoms/IMD example data. RMS 22 may include a collection of modular services for medical diagnosis, treatment, and health monitoring.

In addition, the data from IMD 2 may be provided to clinicians 26 via clinician computing devices 24. Clinician computing devices 24 may include desktop computers, laptops, virtualized computing environments, tablet computers, smart phones, and other computing devices. Clinicians 26 may view patient data generated by IMD 2 on clinician computing devices 24 and analyze the nature of the medical event experienced by patient 4. Additionally, clinicians 26 may include medical technicians at a medical data center who review and assess medical device data for classification.

In an example, patient 4 experiences a medical event such as an arrhythmia or worsening heart failure. IMD 2 generates data regarding the medical event and provides it to user computing device 6. User computing device 6 connects to network 10 via access point 12 and provides the data regarding the medical event to RMS 22. RMS 22 processes the data from IMD 2 to determine an appropriate category of medical event. RMS 22 may compare the data received from IMD 2 to data in the categorization database to determine the category of medical event. In some cases, RMS 22 may process the data received from IMD 2 in addition to other data stored in the memory of RMS 22 such as patient records, population-based data, or the like. RMS 22 may process the data using one or more methods such as using rules or a machine learned model or other classifier, and compare a result of the processing, e.g., a classification, to data in the categorization database. Additionally, clinicians 26 may use clinician computing devices 24 to access RMS 22, view the data from IMD 2, and determine the category of medical event. Clinicians 26 may then enter the classification of the medical event via clinician computing devices 24. Once the category of medical event has been determined, RMS 22 may generate a message for user computing device 6. In addition, RMS 22 may determine an appropriate timing information associated with the message using data from the categorization database. For example, the timing information may include time stamp after which the message is no longer valid. Additionally or alternatively, the timing information may determine a start time stamp before which the message is not valid.

Receptive to the receipt of a message, user computing device 6 may store the message in the memory of user computing device 6. User computing device 6 may compare dynamically and/or on a periodic basis the time stamps of the messages stored in the memory of user computing device 6 to the current time determined by user computing device 6. In an example, user computing device 6 receives a message from RMS 22 with an associated time stamp containing a start date and time of October 10 at 12:00 UTC and an expiration stamp of October 20 at 17:00 UTC. User computing device 6 may then compare the current time of user computing device 6 to the start time and expiration time stamp of the message on a periodic basis to determine when the message becomes valid and when the message ceases to be valid.

The addition of time stamps to a message may assist patient 4 in managing messages. The addition of time stamps may reduce the number of active messages displayed to patient 4 and reduce the risk of more urgent messages being buried beneath less important messages displayed in the GUI of user computing device 6.

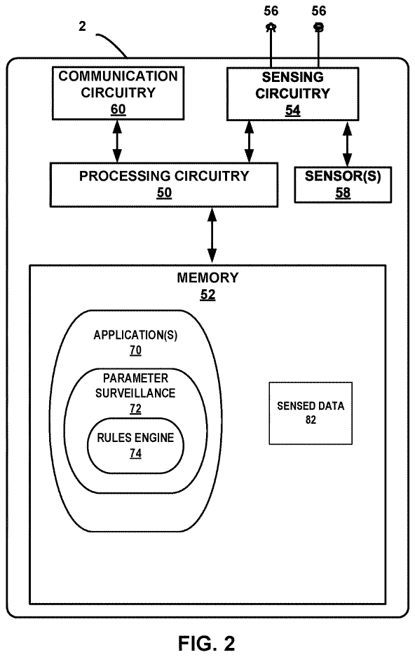

FIG. 2 is a block diagram illustrating an example configuration of IMD 2 of FIG. 1. As shown in FIG. 1, IMD 2 includes processing circuitry 50, memory 52, sensing circuity 54, coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any non-transitory, volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may measure impedance, e.g., of tissue proximate to IMD 2, via electrodes 56. The measured impedance may vary based on respiration, cardiac pulse or flow, and a degree of perfusion or edema. Processing circuitry 50 may determine patient metrics relating to respiration, fluid retention, cardiac pulse or flow, perfusion, and/or edema based on the measured impedance.

Sensing circuitry 54 may also monitor signals from electrodes 56 to, for example, monitor electrical activity of a heart of patient 4 and produce sensor data for patient 4. In some examples, processing circuitry 50 may identify features of the sensed ECG, such as heart rate, heart rate variability, T-wave alternans, intra-beat intervals (e.g., QT intervals), and/or ECG morphologic features, to detect an episode of cardiac arrhythmia of patient 4.

In some examples, IMD 2 includes one or more sensors 58, such as one or more accelerometers, gyroscopes, microphones, optical sensors, temperature sensors, pressure sensors, and/or chemical sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52. Patient parameters determined from signals from sensors 58 may include intravascular fluid level, interstitial fluid level, oxygen saturation, glucose level, stress hormone level, heart sounds, body motion, activity intensity, sleep duration, sleep quality, body posture, or blood pressure.

Memory 52 may store applications 70 executable by processing circuity 50. Applications 70 may include a parameter surveillance application 72. Processing circuitry 50 may execute parameter surveillance application 72 to generate a dataset of a series of patient parameters for diagnosis by RMS 22, as described herein, which may be stored as sensed data 82. Applications 70 may apply rules engine 74 to process sensed data, for example applications 70 may process sensed data 82 to filter data indicate of a medical condition. Rules engine 74 may include one or more models, algorithms, decision trees, and/or thresholds. In some cases, rules engine 74 may be developed based on machine learning, e.g., may include one or more machine learning models.

When parameter surveillance application 72 generates a segment of sensed data 82, parameter surveillance application may store sensed data 82 in memory 52. Sensed data 82 may be provided in a transmission to user computing device 6. The transmission to user computing device 6 may include unprocessed patient data directly from sensed data 82, patient data processed by applications 70, and/or the outcome of the application of rules engine 74 (e.g., a detected cardiac event, a drop in blood glucose level, or other such medical event). Further, the transmission to user computing device 6 may include a message and associated timing information generated by IMD 2. Transmission of the message may occur on an ad hoc basis and as quickly as possible. Communication circuitry 60 may include any suitable hardware, firmware, software, or any combination thereof for wirelessly communicating with another device, such as patient computing device 6. IMD 2 may include elements not illustrated in FIG. 2 such as therapy delivery circuitry (e.g., electrical stimulation circuitry, drug pump circuitry, and other types of circuitry that may be capable of delivering therapy).

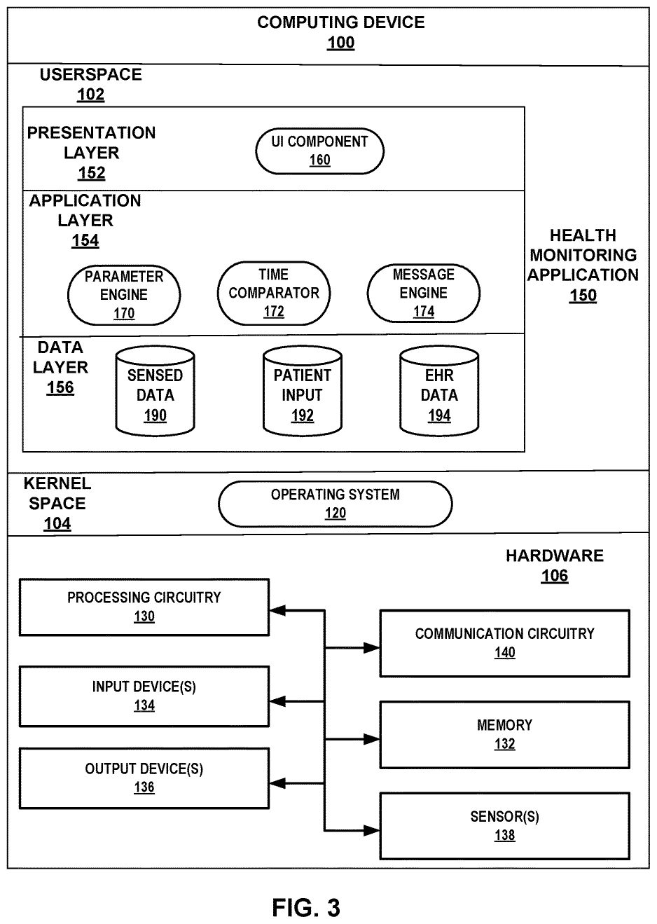

FIG. 3 is a block diagram illustrating an example configuration of patient computing device 100 of patient 4, which may correspond to user computing device 6 illustrated in FIG. 1. In some examples, computing device 100 takes the form of a smartphone, a laptop, a tablet computer, a personal digital assistant (PDA), a dedicated patient monitor, a smartwatch, or other wearable computing device. Although described herein primarily in the context of examples in which the user computing device 6 that receives messages according to the techniques of this disclosure is a device of patient 4, other user computing devices of other users, e.g., caregivers, family members, or clinicians, may similarly implement the techniques of this disclosure.

As shown in the example of FIG. 3, computing device 100 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 100 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 100. For example, processing circuitry 130 may be configured to receive and process instructions stored in memory 132 that provide functionality of components included in kernel space 104 and user space 102 to perform one or more operations in accordance with techniques of this disclosure.

Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 100, for processing during operation of computing device 100. Memory 132, in some examples, is described as a non-transitory computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 134 of computing device 100 may receive input, e.g., from patient 4, clinicians 26, or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 100 may generate output, e.g., to patient 4 or another user. Examples of output are tactile, haptic, audio, and visual output. Output devices 134 of computing device 100 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensors 138 of computing device 100 may sense physiological parameters or signals of patient 4. Sensor(s) 138 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry (e.g., including an ADC), like those described above with respect to IMD 2 and FIG. 2.

Communication circuitry 140 of computing device 100 may communicate with other devices by transmitting and receiving data. Communication circuitry 140 may receive data from IMD 2, such as patients metrics and/or higher resolution diagnostic information, from communication circuitry in IMD 2. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

As shown in FIG. 3, health monitoring application 150 executes in user space 102 of computing device 100. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, parameter engine 170, time comparator 172, and message engine 174. Parameter engine 170 may be responsive to receipt of a transmission from IMD 2 indicating that sensed data 190 from IMD 2 has been received and begin generating data for transmission to RMS 22. Sensed data 190 may include data received from IMD 2, such as patient metrics.

Patient input 192 may include responses to queries posed by health monitoring application 150 or clinicians 26 regarding the condition of patient 4, input by patient 4 or another user. The queries and responses may occur responsive to the generation of data consistent with a medical event or may have occurred prior to the generation, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of SCA, tachyarrhythmia, myocardial infarction, stroke, seizure, one or more disease states, such as status of HF, degree of heart recovery after intervention, COPD, renal dysfunction, or hypertension, aspects of disease state, such as ECG characteristics, cardiac ischemia, oxygen saturation, lung fluid, activity, or metabolite level, genetic conditions, congenital anomalies, history of procedures, such as ablation or cardioversion, and healthcare utilization. EHR data 194 may also include cardiac indicators, such as ejection fraction and left-ventricular wall thickness. EHR data 194 may also include demographic and other information of patient 4, such as age, gender, race, height, weight, and BMI. EHR data 194 may additionally include data from wearable devices such as smartwatches, fitness trackers, and other wearables. Further, EHR data 194 may include medical and health information stored on a computing device such as information from a fitness tracking application or from a health tracking application executing on the computing device.

Application layer 154 provides an execution environment for parameter engine 170, time comparator 172, and message engine 174. Application layer 154 may facilitate access to hardware 106 of computing device 100. Additionally, application layer 154 may provide data such as the sensed data 190, patient input 192, and EHR data 194 to parameter engine 170, time comparator 172, and message engine 174. Application layer 154 may coordinate the operation of parameter engine 170, time comparator 172, and message engine 174 in processing data and messages.

Message engine 174 may receive and process messages and messages received from RMS 22. Message engine 174 may receive packaged messages that contain data such as the category of medical event, timing information (e.g., a start time stamp and/or an end time stamp), and an urgency flag. The category of medical event may consist of a high-level categorization of a medical event, e.g., heart attack or arrhythmia, or a detailed categorization of the event, e.g., ST segment elevation myocardial infarction or polymorphic ventricular tachycardia. The start time stamp may include a time expressed in UTC indicative of when the message from RMS 22 becomes valid for the patient to view. The end time stamp may include a time expressed in UTC indicative of when the message from RMS 22 ceases to be valid for the patient to view. The urgency flag may be used by message engine 174 to determine whether to create a user message containing a warning regarding the urgency of a medical event.

Message engine 174, responsive to the receipt of a message from RMS 22, may generate a visual message for a user in UI component 160. Upon receipt of a message from RMS 22, message engine 174 may provide any attached time stamps to time comparator 172. Time comparator 172 determines the current time (e.g., UTC) and compares start and/or end times stamps to the current time. Time comparator 172 may access a database of messages and associated time stamps that have been received from message engine 174 and located in memory 132. Time comparator 172, comparing time stamps to the current time, determines whether a start time indicated by a start time stamp, if included, has been reached. If there is no start time stamp, message engine 174 generates the visual message immediately upon reception. Additionally, time comparator 172, comparing time stamps to the current time, determines whether a stop time indicated by an end time stamp, if included, has been reached. UI component 160 generates and renders user interfaces of health monitoring application 150 based on the timing information such that only relevant messages are presented to the patient or other user. Time comparator 172, upon determining that a start time of a message has been reached, may cause UI component 160 to update the UI of computing device 100 with a message of a medical event for patient 4. Additionally, time comparator 172, upon determining that the end time of a message has been reached, may cause UI component 160 to update the UI of computing device 100 to remove the message about a medical event from the view of patient 4. In this manner, computing device 100 manages messages to patients from their IMD and health provider by reducing the total number of messages and ensuring that only relevant messages and messages are shown.

UI component 160 of health monitoring application 150 may cause of the UI of computing device 100 to update with a message for patient 4. The message for patient 4 may include a description of the medical event, when the medical event was first detected by IMD 2, the seriousness of the medical event, a banner or other such UI element indicating a heightened severity/urgency of the medical condition, and other related information. Additionally, message engine 174 may adjust the message in response to updates from time comparator 172. In an example, time comparator 172 determines that the current time has exceeded the time stamp of a message. Responsive to the determination, message engine 174 causes UI component 160 to update with the message placed in a “history” of messages rather than to be displayed as an active message or modified to remove the banner or other such UI element indicating heightened severity/urgency but still displayed as an informational message. Further, message engine 174 may store messages that are no longer relevant in memory 132 as a record of messages to patient 4.

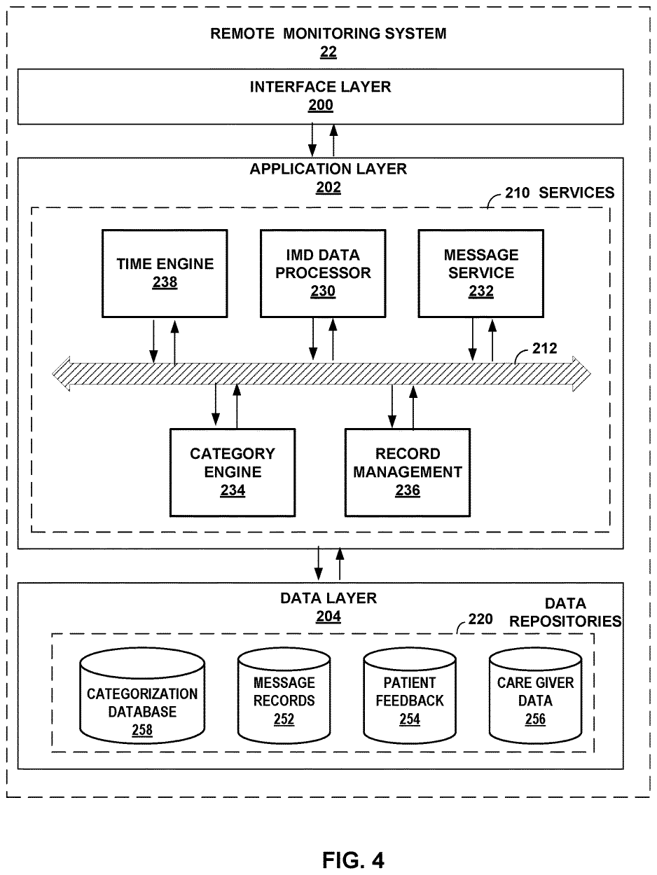

FIG. 4 is a block diagram illustrating an operating perspective of RMS 22. RMS 22 may be implemented in a computing system 20, which may include hardware components such as those of user computing device 6, e.g., processing circuity, memory, and communication circuitry embodied in one or more physical devices. FIG. 4 provides an operating perspective of RMS 22 when hosted as a cloud-based platform. In the example of FIG. 4, components of RMS 22 are arranged according to multiple logical layer that implemented the techniques of this disclosure. Each layer may be implemented by one or more modules comprised of hardware, software, or a combination of hardware and software.

Computing devices, such as clinician devices 24 and user computing device 6 operate as clients that communicate with RMS 22 via interface layer 200. The computing devices typically execute client software applications, such as desktop application, mobile application, and web applications. Interface layer 200 represents a set of application programming interfaces (API) or protocol interfaces presented and supported by RMS 22 for the client software applications. Interface layer 200 may be implemented with one or more web servers.

As shown in FIG. 4, RMS 22 also includes an application layer 202 that represents a collection of services 210 for implementing the functionality ascribed to RMS 22 herein. Application layer 202 receives information from client applications, e.g., sensed data from user computing device 6, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents logical interconnections or set of interfaces that allows different services 210 to send messages to other services, such as by a publish/subscription communication model.

Data layer 204 of HMS 22 provides persistence for information in RMS 22 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within RMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads, or software instructions generally for execution on one or more physical processors.

IMD data processor 230 may be responsive to receipt of patient metrics and other medical device information from IMD 2 via user computing device 6. IMD data processor 230 may initiate analysis of the medical device information transmitted from IMD 2. IMD data processor 230 pre-process and filters data received from IMD 2, e.g., in the case of signals sensed by IMD 2, before providing it to category engine 234. IMD data processor 230 may pre-process data received from IMD 2 to remove undesirable elements such a noise in the data and to organize the data into a standardized format for processing by category engine 234. Category engine 234 receives the medical device data from IMD data processor 230. Category engine 234 may utilize message categories, e.g., medical event categories and associated symptoms/patient metrics, stored in categorization database 258 to determine the message category for a message to delivery to a user, e.g., the patient. Category engine 234 may additionally use data received from user computing device 6 such as weight, age, demographics, EMR, data from clinicians, data provided by a hospital, and other such data in determining the medical event or condition experienced by patient 4. Categorization database 258 may include a plurality of medical events and conditions and patient metrics associated with such events and conditions. For example, categorization database 258 may contain a plurality of example ECG readouts that are consistent with a patient experiencing a heart attack. Category engine 234 may acquire the plurality of example ECG readouts from categorization database 258 and compare the example ECG readouts with an ECG readout received from IMD 2 to determine the likelihood that a patient is experiencing a heart attack or arrhythmia. In an additional example, IMD 2 provides to RMS 22 medical data over a period of several days that is consistent with an increasing risk of heart failure in patient 4. Category engine 234 may utilize the data generate heart failure risk scores. Additionally, category engine 234 may provide the data to one or more services not illustrated such as a risk stratification tool like TriageHF™ risk stratification tool commercially available from Medtronic plc, of Dublin, Ireland to determine whether patient 4 is experiencing a medical event such as HF decompensation. The one or more services may determine whether patient 4 is experiencing a medical event based on whether one or more patient metrics have been exceeded. Category engine 234, responsive to the determination that patient 4 is at an elevated risk of heart failure may provide the determination to message service 232. Message service 232 may generate and provide a message to user device 6 regarding the elevated risk of heart failure. Patient 4 may configure how they receive the messages from message service 232 (e.g., email, text message, message from an application on a computing device).

Category engine 234, having determined the category of medical event, may utilize time engine 238 to determine the length of time the message to a patient should remain valid. Time engine 238 may use the category of event to determine the length of time the message should remain valid. Time engine 238 may utilize the time at which the message is to be sent to user computing device 6 to determine a start time stamp and an end time stamp for the message if applicable to the category of medical event. Time engine 238 may use recommended time durations associated with medical events stored in categorization database 258 to determine the applicable start and end time stamps for the message. In an example, category engine 234 determines that patient 4 is exhibiting worsening symptoms of heart failure. Category engine 234 may utilize time engine 238 to determine a time duration for which the message to patient 4 should remain valid. In this example, time engine 238 may determine that, due to the severity and worsening nature of the heart failure, the message should remain active indefinitely or until patient 6 is seen by a medical professional. In an additional example, patient 6 has been diagnosed with heart failure but is exhibits only a brief worsening of symptoms followed by a period of improvement. In the example, time engine 238 may determine that the message need only remain valid for a period of time as long as the brief worsening of symptoms does not reoccur. In another example, category engine 234 may generate a message with a start time stamp that indicates when the message should become visible on user computing device 6. In such an example, it may be desirable for a message to not be immediately visible to patient 4 (e.g., a message that is part of a recurring series of messages to a user such as reminder to schedule a new appointment that does not appear until after the date of the prior appointment).

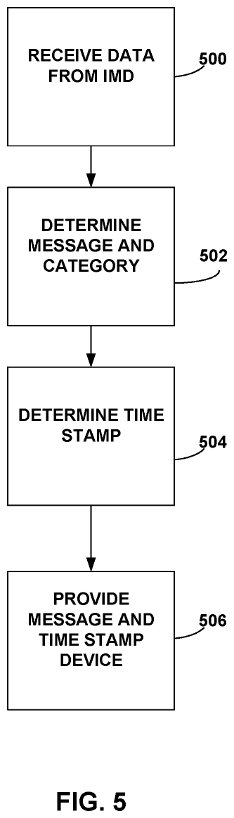

FIG. 5 is a flow diagram illustrating an example operation of RMS 22 receiving data from an IMD, determine a category of medical event that correlates with the received data, determine a time duration for which a message to patient 4 is visible, generating a message for user computing device 6, and providing the message to user computing device 6. The example operation of FIG. 6 may be performed the by processing circuity and communication circuity of computing system 20, and by extension computing system 100. Furthermore, although described in the context of an example in which RMS 22 determines and provides messages based on medical device data generated by an IMD, in some examples, other devices, such as the IMD, may similarly perform the example operation of FIG. 5.

As seen in the example of FIG. 5, processing circuity in IMD 2 may generate data regarding patient metrics and provide it to RMS 22. IMD 2 may provide the data to RMS 22 by wireless communications with user computing device 6, which connects to RMS 22 via network 10 (500). Following receipt of the data generated by IMD 2, RMS 22 provides the patient data to event categorizer 234 for categorization of the medical event. Category engine 234 compares the received patient data with categorization data from categorization database 258 to determine a category of medical event and a message (502). Category engine 234 may use at least one of a plurality of algorithms or machine learning techniques to determine the medical event category to associate with the data received from IMD 2. Category engine 234 uses data from categorization database 258 to determine the time duration the message should be visible patient 4 on user computing device 6 (504). Category engine 234 uses the recommended time periods to determine the length of the time the message should remain visible to patient 4. Additionally, category engine 234 determines, based on the nature of the medical event, whether an urgency flag will be included with the message. Category engine 234 may package the data regarding the category of medical event, the start and end time stamps (if applicable) and any urgency flags. Category engine 234 provides the packaged data regarding the medical event to message service 232 for transmission to user computing device 6. Message service 232 executing on RMS 22 provides the packages data to user computing device 6 (506).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system including at least one device comprising memory configured to store medical device data generated by a medical device of a patient; and one or more programmable processors in communication with the memory and configured to determine, based on the medical device data, a message for a user and an expiration time stamp for the message after which the message is no longer valid; and provide the message including the expiration time stamp to a computing device of the user.

Example 2: The system of example 1, wherein the at least one device comprises at least one server device associated with a remote medical system, the at least one server device further comprising a network communication interface, wherein the one or more programmable processors are in communication with the network communication interface and configured to receive the medical device data from the medical device via the network communication interface; determine, based on the medical device data, a message for a user and an expiration time stamp for the message after which the message is no longer valid; and provide the message including the expiration time stamp to the user computing device via the network communication interface.

Example 3: The system of example 1, wherein the at least one device comprises the medical device, wherein the medical device comprises: communication circuitry configured to wirelessly communicate with the user computing device; and at least one of sensing circuitry configured to sense one or more physiological parameters of the patient or therapy delivery circuitry configured to deliver one or more therapies to the patient, wherein the one or more programmable processors are configured to: generate the medical device data based on at least one of the physiological parameters or the therapies; and provide the message including the expiration time stamp to the user computing device via the communication circuitry.

Example 4: The system of any one or more of examples 1 to 3, wherein the medical device comprises an implantable medical device (IMD).

Example 5: The system of any one or more of examples 1 to 4, wherein to determine the expiration time stamp for the message, the one or more programmable processors are configured to determine one message category for the message from a plurality of message categories; and determine the expiration time stamp based on the message category.

Example 6: The system of any one or more of examples 1 to 4, wherein to determine the message and the expiration time stamp, the one or more programmable processors are configured to compare the medical device data with criteria for a plurality of categories located in a categorization database, wherein the categorization database comprises a database of a plurality of message, messages categories associated with the plurality of messages, and time stamps associated with the plurality of message categories.

Example 7: The system of example 6, wherein at least one of the criteria and expiration time stamps are programmable for the patient by a clinician.

Example 8: The system of any one or more of examples 1 to 7, wherein the message is configured to cause the user computing device to determine, based on a comparison of a length of time elapsed since the message has been received and the expiration time stamp of the message, the message is no longer valid; and modify, via a user interface of the user computing device, the visibility of the message such that the message is associated with non-urgent communications or ceases to be visible to a user based on the determination that the message is no longer valid.

Example 9: The system of any one or more of examples 1 or 8, wherein the expiration time stamp is a first time stamp, and wherein, to generate the message regarding the medical device data, the one or more programmable processors are configured to generate a flag indicating a high urgency of the message, and include a second time stamp after which the flag indicating a high urgency of the message is no longer valid.

Example 10: The system of example 9, wherein the flag indicating a high urgency of the medical device data causes the user computing device to generate, via a user interface of the user computing device, a notification to the user indicating the high urgency of the message; and modify, via the user interface, the notification to the user to no longer indicate the high urgency of the message based on a comparison of a length of time elapsed since the message has been received and the second time stamp of the message.

Example 11: The system of any one or more of examples 1 to 10, wherein the one or more programmable processors are configured to generate the message to further include a start time stamp where the message is not valid until the time indicated by the start time stamp is reached based on the medical device data.

Example 12: The system of example 11, wherein the message including the start time stamp causes the user computing device to determine, based on a comparison of the current time being greater than the start time stamp of the message, the message is valid; and modify, via the user interface of the user computing device, the visibility of the message such that the message becomes visible to the user in response to the determination.

Example 13: The system of any one or more of examples 1 to 12, wherein the medical device data includes one or more physiological parameters of the patient sensed by the medical device.

Example 14: The system of any one or more of examples 1 to 13, wherein the medical device data includes data indicating one or more therapies delivered to the patient by the medical device.

Example 15: The system of any one or more of examples 1 to 14, wherein the medical device data includes data indicating an operational parameter of the medical device.

Example 16: The system of any one or more of examples 1 to 15, wherein the medical device data includes data indicating detection of a medical event by the medical device.

Example 17: The system of example 16, wherein the medical event comprises an arrhythmia.

Example 18: The system of any one or more of examples 1 to 17, wherein the message comprises one or more survey questions for a patient.

Example 19: The system of any one or more of examples 1 to 18, wherein the one or more programmable processors are configured to determine, based on the medical device data, a medical event of the patient, and select the message and expiration time stamp based on the medical event.

Example 20: The system of example 19, wherein the medical event comprises an arrhythmia.

Example 21: The system of any one of examples 19 or 20, wherein the medical event comprises a heart failure event.

Example 22: The system of any one or more of examples 19 to 21, wherein the one or more programmable processors are configured to determine at least one of a severity or a likelihood of the medical event, and select the message and expiration time stamp based on the at least one of the severity or the likelihood of the medical event.

Example 23: A method comprising determining, based on medical device data generated by a medical device of a patient, a message for a user and an expiration time stamp for the message after which the message is no longer valid; and providing the message including the expiration time stamp to a user computing device of the user.

Example 24: The method of example 23, further comprising receiving, by at least one server device associated with a remote medical system the medical device data from the medical device, wherein the at least one server device determines the message and the expiration time stamp and provides the message and the expiration time stamp to the user computing device.

Example 25: The method of example 23, wherein the medical device determines the message and the expiration time stamp and provides the message and the expiration time stamp to the user computing device via wireless communication with the user computing device.

Example 26: The method of any one or more of examples 23 to 25, wherein the medical device comprises an implantable medical device (IMD).

Example 27: The method of any one or more of examples 23 to 26, wherein determining the expiration time stamp for the message comprises determining one message category for the message from a plurality of message categories; and determining the expiration time stamp based on the message category.

Example 28: The method of any one or more of examples 23 to 26, wherein determining the message and the expiration time stamp for the message comprises comparing the medical device data with criteria for a plurality of categories located in a categorization database, wherein the categorization database comprises a database of the plurality of messages, messages categories associated with the plurality of messages, and time stamps associated with the plurality of message categories.

Example 29: The system of method of example 27, wherein at least one of the criteria and expiration time stamps are programmable for the patient by a clinician.

Example 30: The method of any one or more of examples 23 to 29, wherein the message is configured to cause the user computing device to determine, based on a comparison of a length of time elapsed since the message has been received and the expiration time stamp of the message, the message is no longer valid; and modify, via a user interface of the user computing device, the visibility of the message such that the message is associated with non-urgent communications or ceases to be visible to a user based on the determination that the message is no longer valid.

Example 31: The method of any one or more of examples 23 to 30, wherein the expiration time stamp is a first time stamp, and wherein generating the message comprises generating a flag indicating a high urgency of the message; and including a second time stamp after which the flag indicating a high urgency of the medical event is no longer valid.

Example 32: The method of example 31, wherein the flag indicating a high urgency of the medical device data causes the user computing device to generate, via a user interface of the user computing device, a message to the user regarding the high urgency of the message; and modify, via the user interface, the notification to the user to no longer indicate the high urgency of the message based on a comparison of a length of time elapsed since the message has been received and the second time stamp of the message.

Example 33: The method of any one or more of examples 23 to 32, wherein the message regarding a medical event includes a start time stamp where the message is not valid until the time indicated by the start time stamp is reached based on the medical device data.

Example 34: The method of example 33, wherein the message including the start time stamp causes the user computing device to determine, based on a comparison of the current time being greater than the start time stamp of the message, the message is valid; and
modify, via the user interface of the user computing device, the visibility of the message such that the message becomes visible to the user in response to the determination.

Example 35: The method of any one or more of examples 23 to 34, wherein the medical device data includes one or more physiological parameters of the patient sensed by the medical device.

Example 36: The method of any one or more of examples 23 to 35, wherein the medical device data includes data indicating one or more therapies delivered to the patient by the medical device.

Example 37: The method of any one or more of examples 23 to 36, wherein the medical device data includes data indicating an operational parameter of the medical device.

Example 38: The method of any one or more of examples 23 to 37, wherein the medical device data includes data indicating detection of a medical event by the medical device.

Example 39: The method of example 38, wherein the medical event comprises an arrhythmia.

Example 40: The method of any one of examples 23 to 39, wherein the message comprises one or more survey questions for the patient.

Example 41: The method of any one or more of examples 23 to 40, further comprising determining, based on the medical device data, a medical event of the patient; and selecting the message and expiration time stamp based on the medical event.

Example 42: The method of example 41, wherein the medical event comprises an arrhythmia.

Example 43: The method of any one of examples 41 or 42, wherein the medical event comprises a heart failure event.

Example 44: The method of any one or more of examples 41 to 43, further comprising determining at least one of a severity or a likelihood of the medical event; and selecting the message and expiration time stamp based on the at least one of the severity or the likelihood of the medical event.

Example 45: A non-transitory computer-readable storage device comprising instructions for causing processing circuitry to determine, based on medical device data generated by a medical device of a patient, a message for a user and an expiration time stamp for the message after which the message is no longer valid; and provide the message including the expiration time stamp to a user computing device of the user.

Example 46: The non-transitory computer-readable storage device of example 45, wherein the message is configured to cause the user computing device to determine, based on a comparison of a length of time elapsed since the message has been received and the expiration time stamp of the message, the message is no longer valid; and modify, via a user interface of the user computing device, the visibility of the message such that the message is associated with non-urgent communications or ceases to be visible to a user based on the determination that the message is no longer valid.

Example 47: A computing device comprising communication circuitry configured to receive a message and an expiration time stamp for the message from another device, wherein the expiration time stamp indicates a time after which the message is no longer valid, the message and the expiration time stamp determined by the other device based on medical device data of a medical device of a patient; a user interface; and one or more programmable processors configured to control presentation of the message via the user interface based on the expiration time stamp.

Example 48: The computing device of example 47, wherein the message is configured to cause the one or more programmable processors to determine, based on a comparison of a length of time elapsed since the message has been received and the expiration time stamp of the message, the message is no longer valid; and modify the presentation of the message via the user interface such that the message is associated with non-urgent communications or ceases to be visible to a user based on the determination that the message is no longer valid.

Example 49: The computing device of examples 47 or 48, wherein the message includes a flag indicating a high urgency of a medical event and a second time stamp after which the flag indicating the high urgency of the medical event is no longer valid.

Example 50: The computing device of example 49, wherein the one or more programmable processors are configured to generate, via the user interface, a message to the user indicating the high urgency of the medical event; and modify, via the user interface, the message to the user to no longer indicate the high urgency of the medical event based on a comparison of a length of time elapsed since the message has been received and the second time stamp of the message.

Example 51: The computing device of any one or more of examples 47 to 50, wherein the message comprises one or more survey questions for the patient.

1. A system including at least one device comprising:
memory configured to store medical device data generated by a medical device of a patient; and
one or more programmable processors in communication with the memory and configured to:
determine, based on the medical device data, a message for a user and an expiration time stamp for the message after which the message is no longer valid; and
provide the message including the expiration time stamp to a user computing device.

2. The system of claim 1, wherein the at least one device comprises at least one server device associated with a remote medical system, the at least one server device further comprising a network communication interface, wherein the one or more programmable processors are in communication with the network communication interface and configured to:
receive the medical device data from the medical device via the network communication interface;
determine, based on the medical device data, the message for the user and the expiration time stamp; and
provide the message including the expiration time stamp to the user computing device via the network communication interface.

3. The system of claim 1, wherein the at least one device comprises the medical device, wherein the medical device comprises:
communication circuitry configured to wirelessly communicate with the user computing device; and
at least one of sensing circuitry configured to sense one or more physiological parameters of the patient or therapy delivery circuitry configured to deliver one or more therapies to the patient, wherein the one or more programmable processors are configured to:
generate the medical device data based on at least one of the physiological parameters or the therapies; and
provide the message including the expiration time stamp to the user computing device via the communication circuitry.

4. The system of claim 1, wherein to determine the expiration time stamp for the message, the one or more programmable processors are configured to:
determine one message category for the message from a plurality of message categories; and
determine the expiration time stamp based on the message category.

5. The system of claim 1, wherein to determine the message and the expiration time stamp, the one or more programmable processors are configured to:
compare the medical device data with criteria for a plurality of categories located in a categorization database, wherein the categorization database comprises a database of a plurality of messages, messages categories associated with the plurality of messages, and time stamps associated with the plurality of message categories.

6. The system of claim 1, wherein the message is configured to cause the user computing device to:
determine, based on a comparison of a length of time elapsed since the message has been received and the expiration time stamp of the message, the message is no longer valid; and
modify, via a user interface of the user computing device, the visibility of the message such that the message is associated with non-urgent communications or ceases to be visible to a user based on the determination that the message is no longer valid.

7. The system of claim 1, wherein the expiration time stamp is a first time stamp, and wherein, to generate the message regarding the medical device data, the one or more programmable processors are configured to:
generate a flag indicating a high urgency of the message; and
include a second time stamp after which the flag indicating a high urgency of the message is no longer valid.

8. The system of claim 7, wherein the flag indicating a high urgency of the medical device data causes the user computing device to:
generate, via a user interface of the user computing device, a notification to the user indicating the high urgency of the message; and
modify, via the user interface, the notification to the user to no longer indicate the high urgency of the message based on a comparison of a length of time elapsed since the message has been received and the second time stamp of the message.

9. The system of claim 1, wherein the one or more programmable processors are configured to generate the message to further include a start time stamp where the message is not valid until the time indicated by the start time stamp is reached based on the medical device data.

10. The system of claim 9, wherein the message including the start time stamp causes the user computing device to:
determine, based on a comparison of the current time being greater than the start time stamp of the message, the message is valid; and
modify, via the user interface of the user computing device, the visibility of the message such that the message becomes visible to the user in response to the determination.

11. The system of claim 1, wherein the medical device data includes one or more physiological parameters of the patient sensed by the medical device, data indicating one or more therapies delivered to the patient by the medical device, data indicating an operational parameter of the medical device, or data indicating detection of a medical event by the medical device.

12. The system of claim 1, wherein the message comprises one or more survey questions for a patient.

13. The system of claim 1, wherein the one or more programmable processors are configured to determine, based on the medical device data, a medical event of the patient, and select the message and expiration time stamp based on the medical event.

14. The system of claim 13, wherein the medical event comprises one of an arrhythmia or a heart failure event.

15. The system of claim 13, wherein the one or more programmable processors are configured to determine at least one of a severity or a likelihood of the medical event, and select the message and expiration time stamp based on the at least one of the severity or the likelihood of the medical event.

16. A method, comprising:
determining, based on medical device data generated by a medical device of a patient, a message for a user and an expiration time stamp for the message after which the message is no longer valid; and
providing the message including the expiration time stamp to a user computing device of the user.

17. The method of claim 16, further comprising receiving, by at least one server device associated with a remote medical system the medical device data from the medical device, wherein the at least one server device determines the message and the expiration time stamp and provides the message and the expiration time stamp to the user computing device.

18. The method of claim 16, wherein the medical device determines the message and the expiration time stamp and provides the message and the expiration time stamp to the user computing device via wireless communication with the user computing device

19. The method of claim 16, wherein the medical device comprises an implantable medical device (IMD).

20. A non-transitory computer-readable storage device comprising instructions for causing processing circuitry to:
determine, based on medical device data generated by a medical device of a patient, a message for a user and an expiration time stamp for the message after which the message is no longer valid; and
provide the message including the expiration time stamp to a user computing device of the user.
